(19) United States
(12) Patent Application Publication
(10) Pub. No.: US 2008/0052786 A1
(43) Pub. Date: Feb. 28, 2008

(54) Animal Model of Prostate Cancer and Use Thereof

(76) Inventors: Pei-Cheng Lin, Taichung (TW); Han-Hsin Chang, Taichung (TW); Bo-Yie Chen, Taichung (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/467,147

(22) Filed: Aug. 24, 2006

(51) Int. Cl. A01K 67/027

(52) U.S. Cl. 800/10; 800/14

(57) ABSTRACT

The present invention relates to an adult mammal which exhibits growth or replication of abnormal cells in a target tissue or organ by over-expressing Hedgehog protein in such target tissue or organ. The present invention also relates to a method of preparing an adult animal model of prostate cancer. The invention further relates to a method of evaluating an agent for treating prostate cancer.

### FIELD OF THE INVENTION

The present invention relates to a mammal susceptible to prostate cancer, a method of preparing a animal model of prostate cancer, and a method of evaluating an agent for treating prostate cancer.

### BACKGROUND OF THE INVENTION

Prostate cancer, apart from skin cancer, is the most common male malignancy and the second leading cause of cancer deaths in men in the United States (R. T. Greenlee, T. Murray, S. Bolden, P. A. Wingo, CA Cancer J Clin 50, 7 (2000)). It had surpassed lung cancer in 1990 and was estimated to cause 31,000 deaths in 2002 in the United States alone (W. Isaacs, A. De Marzo, W. G. Nelson, Cancer Cell 2, 113 (2002)). Despite the increasing incidence, prostate cancer presents some obstacles that hind clinicians and basic researchers from understanding its pathogenesis. prostate cancer is characterized by slow clinical progression, involvement of multiple genetic and epigenetic events, multifocal and heterogeneous nature of tumorigenesis, and inability to determine prognosis for disease progression (A. M. De Marzo et al., Urology 62, 55 (2003); C. Abate-Shen, M. M. Shen, Trends Genet 18, S1 (2002)). Given the above, mouse models are advantageous for studying prostate cancer, despite intrinsically anatomical differences and probably different molecular mechanisms underlying prostate carcinogenesis (C. Abate-Shen, M. M. Shen, Trends Genet 18, S1 (2002); W. J. Huss, L. A. Maddison, N. M. Greenberg, Semin Cancer Biol 11, 245 (2001)).

Several types of mouse models have been established, such as reconstitution models, xenograft models, hormonal models, as well as transgenic and knockout models (N. M. Navone, C. J. Logothetis, A. C. von Eschenbach, P. Troncoso, Cancer Metastasis Rev 17, 361 (1998); C. Abate-Shen, M. M. Shen, Genes Dev 14, 2410 (2000)). Conceivably, an ideal model has to exhibit characteristics closely analogous to those found in the human disease. However, various deviations have been observed in the mouse models. For example, the rat probasin promoter was used to drive the expression of SV40 large T and small t tumor antigens, producing the TRAMP (transgenic adenocarcinoma mouse prostate) prostate cancer model or, if without the small t antigen, the LADY model (W. J. Huss, L. A. Maddison, N. M. Greenberg, Semin Cancer Biol 11, 245 (2001); N. M. Navone, C. J. Logothetis, A. C. von Eschenbach, P. Troncoso, Cancer Metastasis Rev 17, 361 (1998)). These transgenic models, also called SV40-Tag models, displayed neuroendocrine features that are only seen in about 10% of human cases (J. H. Park et al., Am J Pathol 161, 727 (2002); N. Masumori et al., J Urol 171, 439 (2004)). Besides, the SV4-Tag models usually develop high-grade PIN (prostate intraepithelial neoplasia) within 12 to 20 weeks of age, followed by subsequent metastases at 30 weeks (J. R. Gingrich, R. J. Barrios, B. A. Foster, N. M. Greenberg, Prostate Cancer Prostatic Dis 2, 70 (1999)). Such aggressive progression is not in proportion to the status of human disease where 40 or more years are usually needed to progress from benign prostatic hyperplasia (BPH) or PIN to detectable prostate cancer. Another example of deviation is shown in some mouse transgenic or knockout models produced without using the SV40-Tag (C. Abate-Shen, M. M. Shen, Trends Genet 18, S1 (2002)). The non-SV40-Tag models displayed high proportions of atypical epithelial lesions representing different degrees of PIN without frequent progression into invasive carcinoma, which makes it difficult to prove their malignant potential (J. H. Park et al., Am J Pathol 161, 727 (2002)). These deviations do not necessarily devaluate the usage of mouse models, but reflect the complexity of mammalian prostate tumorigenesis and, at the same time, a demand for more animal models.

Sonic hedgehog (Shh) was originally identified as a homologue of the hedgehog segment-polarity gene of Drosophila, along with two other mammalian homologues, Indian hedgehog (Ihh) and Desert hedgehog (Dhh). Shh has been reported to be involved in many processes during embryogenesis, including dorsoventral patterning of body axis, specifications of neuronal and oligodendrocytic cell fate, axonal outgrowth, cell proliferation, cell differentiation, and cell survival (P. W. Ingham, A. P. McMahon, Genes Dev 15, 3059 (2001)). Recent studies showed a key role of Shh signaling in mediating epithelial-mesenchymal interactions during endoderm-derived tissue formation, including prostate formation. Shh was expressed in the epithelium during prostatic branching morphogenesise and was suggested to involve in the initiation of androgen-dependent prostate development (C. A. Podlasek, D. H. Barnett, J. Q. Clemens, P. M. Bak, W. Bushman, Dev Biol 209, 28 (1999); M. L. Lamm et al., Dev Biol 249, 349 (2002)). More recent data from analyses of Shh mutant fetuses revealed that Shh signaling was not critical for prostatic induction and expression of Shh and its downstream Ptc gene was not regulated by androgens (S. H. Freestone et al., Dev Biol 264, 352 (2003); D. M. Berman et al., Dev Biol 267, 387 (2004)). The prostate defects in Shh mutants could be rescued with androgen supplements, suggesting that Shh signaling acted at least partially through androgen activities (D. M. Berman et al., Dev Biol 267, 387 (2004)).

Like many developmentally critical genes, Shh over-activation has been shown to cause tumorigenesis. Mutations in Ptc gene, a tumor suppressor gene and a Shh signaling pathway repressor, were shown to cause cerebellar medulloblastomas (L. V Goodrich, L. Milenkovic, K. M. Higgins, M. P. Scott, Science 277, 1109 (1997)) and basal cell carcinomas (A. E. Oro et al., Science 276, 817 (1997)) in mice, as well as superficial bladder cancer in human (T. O. Aboulkassim, H. LaRue, P. Lemieux, E Rousseau, Y Fradet, Oncogene 22, 2967 (2003)). Hedgehog signaling has also been reported as required or mediating in the formation of small-cell lung cancer (D. N. Watkins et al., Nature 422, 313 (2003)), pancreatic cancer (S. P. Thayer et al., Nature 425, 851 (2003)), digestive tract tumors (D. M. Berman et al., Nature 425, 846 (2003)), and ameloblastomas (H. Kumamoto, K. Ohki, K. Ooya, J Oral Pathol Med 33, 185 (2004)). Abundant Gli-1 expression was found in 9 of 11 prostate cancer tissues examined, which suggested that Hedgehog signaling could play a role in prostate tumorigenesis (N. Dahmane et al., Development 128, 5201 (2001)). However, so far, there is no Hedgehog pathway gene mutation reported in prostate cancer. More recently, Fan et al. established a xenograft model to elucidate paracrine interactions between Shh-expressing human LNCaP tumor cells and host mouse stromal cells. The genetically engineered Shh-over-expressing LNCaP cells, when subcutaneously co-injected with Matrigel, was shown to increase stromal Gli-1 expression and dramatically accelerate tumor growth (L. Fan et al., Endocrinology 145, 3961 (2004)). Despite these data, there is so far no mouse prostate cancer model caused initially by Hedgehog dysregulation and a potential role of Hedgehog in the initiation and progression of prostate cancer remains to be elucidated.

Accordingly, it would be useful to have a non-human animal model of prostate cancer which can be easily established within a short time.

### SUMMARY OF THE INVENTION

The present invention relates to an adult mammal which exhibits growth or replication of abnormal cells in a target tissue or organ by over-expressing Hedgehog protein in such target tissue or organ. The present invention also relates to a method of preparing an adult animal model of prostate cancer, comprising: (a) introducing a Hedgehog-expressing vector into a prostate of the animal; and (b) expressing the Hedgehog protein in the animal. The invention further relates to a method of evaluating an agent for treating prostate cancer, comprising: (a) administering the agent to be evaluated to an adult animal model of prostate cancer which over-expresses Hedgehog protein in the prostate thereof; and (b) determining the effect of said agent upon a phenomenon associated with prostate cancer.

### BRIEF DESCRIPTION OF THE DRAWINGS

Additional objects and features of the present invention will become more apparent and the invention itself will be best understood from the following Detailed Description of the Invention, when read with reference to the accompanying drawings.

FIG. 1 shows schematic representation of vector injection and electroporation, confirmation of Hedgehog overexpression and its expression patterns, and macro- and microscopic effects of Hedgehog overexpression. (a) The mouse prostate was exposed by surgery and injected through a glass needle with 10 ll of pCX-Shh-IG or pCX-IG at 0.1 lg/ll in 0.9% NaCl, or 0.9% NaCl alone. After injections, the prostate lobes were subjected to electric pulses for 10 s set at 20 volts, 1-2 pulses per second; the mice were then stitched, recovered, and maintained until dissection. (b) Western blot analyses confirmed overexpression of Hedgehog protein tagged with GFP (indicated by arrowhead) that persisted for 90 days after injection. (c) Evident overgrowth with more blood vessels but less lobular formation in the seminal vesicle, was seen in the pCX-shh-IG-injected mouse prostate, as compared to the 0.9% NaCl-injected control in (d) or the pCX-IG-injected control in (e). (f) and (g) Immunodetection of mouse Hedgehog expression using 5E1 antibody following pCX-shh-IG injection. The highly expressed Hedgehog protein was detected in the epithelium as well as in the stroma (indicated by arrows), comparable to that in the human CaP specimen using N-19 antibody for Hedgehog detection in (h) and (i).

FIG. 2 shows the effects of pCX-shh-IG injections at day 30. A to H, I, L: from anterior lobes. J and K: from dorsolateral lobe. Magnifications: A: 20×; B: 40×; C, D, E, I: 100×; F, G, H, I, K, L, 400×. (A) GFP could be detected in whole mounts in the AP (inlet) and in the seminal vesicle (SV). BPH (arrows) and PIN (asterisks) were found in the AP (B, E to I) and DLP (J to K) of pCX-shh-IG injections, in contrast to the pCX-IG injection (C) and the 0.9% NaCl injection (D). BPH and PIN commonly occurred with concomitant stromal hyperplasia and hints of angiogenesis (circled with dotted line in B). CaP could be found at as early as day 30 after injection (L).

FIG. 3 shows the confirmation and localization of Hedgehog expression by immunohistochemical detection with anti-GFP and 5E1 anti-Shh in the pCX-shh-IG injections at day 30. A, B, D, E, G to I: from anterior lobe. C and F: from dorsolateral lobe. Magnifications: A, B, G: 100×; inlets of A, B, G: 400×; B, C, E, F, H, I: 400×. GFP was intensely localized in the hyperplastic stroma (white arrow in A) and the high grade PIN (asterisk in C). Comparable localizations were found in the 5E1 anti-Shh dectections (D, E, F). Both anti-GFP and 5E1 localized basal cells (black arrow) in the pCX-shh-IG injections (inlets of A, D) as well as in the 0.9% NaCl injections detected by 5E1 (inlet of G). No comparable stromal hyperplasia and intense GFP or 5E1 stains were observed in the 0.9% NaCl injections (G). Hedgehog expression remained intense with the appearance of CaP (H, I).

FIG. 4 shows histological effects of Hedgehog overexpression at 30 days after the injection as shown by Hematoxylin-Eosin stain. Prostates with 0.9% NaCl injection in (a) and pCX-IG injection in (b) showed no tumorigenic characteristics. (c) A pCX-shh-IGinjected prostate showed glandular infoldings and stromal hyperplasia. (d) A pCX-shh-IG-injected prostate showed stromal hyperplasia and hypervascularization (indicated by arrows). (e) A pCX-IG-injected prostate showed normal features of epithelial and basal cells. (f-h) pCX-shh-IG-injected prostates showed different levels of PIN formation. Few layers of atypical cells were found in (f), but more layers were deployed in (g) and even more in (h).

FIG. 5 shows cytological effects of Hedgehog overexpression at 30 days after the injection as shown by Hematoxylin-Eosin stain. The arrow-indicated cells were further magnified and presented in the inlets to show cytological changes of the nuclei and the nucleoli. (a) A prostate with 0.9% NaCl injection showed no tumorigenic characteristics. (b) A pCX-shh-IG-injected prostate exhibited PIN formation with cells containing enlarged nuclei and prominent nucleoli. (c) A pCX-shh-IG-injected prostate exhibited the same PIN characteristics as in (b), but with smaller glands and loss of basal cell layer surrounding the arrow-indicated area, suggestive of CaP formation. (d) Mix-up of invasive CaP cells with stromal cells.

FIG. 6 shows immunohistochemical detections of p63 (basal cell marker), α-SMA (fibro-muscular cell marker), and CK-8/CK-18 (epithelial cell marker) for further confirmation of tumorigenic phenotypes induced by Hedgehog overexpression. (a-d) Were from p63 detections; (e) and (f) from CK-8/CK-18 detections; (g) and (h) from α-SMA detections. (a, e and g) Were from 0.9% NaCl-injected prostates; (b-d, f, h) Were from pCX-shh-IG-injected prostates. (a) p63 positive cells (arrow-indicated) showed a normal continuous layer of basal cells. (b) A discontinuous distribution of basal cell layer (arrow-indicated), indicative of PIN formation. (c) A smaller gland, with atypical cells filled the lumen of the duct and loss of p63 positive cells in some foci (arrow-indicated), indicative of invasive CaP formation. (d) A CaP formation with loss of p63 positive cells and absence of fibro-muscular sheath. (e) A normal CK-8/CK-18 positive epithelial distribution. (f) A prostate showed mix-up of epithelial cells in the stroma (arrow-indicated), indicative of invasive CaP formation. (g) A normal thick, dense, and continuous α-SMA positive fibro-muscular sheath. (h) A thin, discontinuous α-SMA positive fibro-muscular sheath following pCX-shh-IG injection.

FIG. 7 shows the Hedgehog-induced prostate tumorigenesis by immunohistochemical detection using E-cadherin, CK14, and p63 as markers, RT-PCR of Hedgehog signaling members, and Western analyses of GFP and PSA at day 30 after the procedure. A to D: from anterior lobe. Magnifications: A to D, 400×. E-cadherin was intensely expressed in the PIN (asterisk), with less expression along the membrane of normal (arrowhead) and BPH (arrow) luminal cells (A). Within the area of CaP, E-cadherin signals were diminished (inlet of A). CK14 was intensely expressed in a displacement and derangement manner in the BPH (arrow) and PIN (asterisk) and was diminished within the area of CaP (B). Another basal cell marker p63 was highly expressed within PIN and CaP (C), as compared to that within BPH (arrows in D) and the 0.9% NaCl-injected tissues (inlet of D). Signs of invasive CaP (arrowheads in C) and basal cell hyperplasia (arrowheads in D) were commonly observed, with loss of p63 stain found in CaP (circled area in D). Increased GFP protein (arrowhead-indicated on upper panel of F) was detected by Western analyses in correlation with elevated serum PSA in two forms (indicated by arrowhead and arrow on lower panel of F). RT-PCR analyses showed elevated expression of Ptc-1, Ptc-2, Gli-1, Gli-2, and Gli-3 (E) with pCX-shh-IG injections.

FIG. 8 shows the immunohistochemical detection of Ptc-1, Gli-1, Gli-2, Gli-3, Hip, and Pten expression at day 30 after injection. A tp L: from anterior lobe. Magnifications: A to L: 400×. Ptc-1 expression was detected in the CaP (A), BPH/PIN epithelial cells, and stromal cells (B), with signals more intense in the pCX-shh-IG injections than those in the normal saline-injected luminal epithelium (inlet of A). Gli-1 was highly expressed in the CaP (C), BPH/PIN epithelial cells, and stromal cells (D), in contrast to lack of evident signal in the epithelium from normal saline injections (inlet of C). Gli-2 expression was found in the CaP (E) and stroma (F), without evident signal in the epithelium from normal saline injections (inlet of E) nor in the BPH/PIN epithelial cells (F). Gli-3 was highly expressed in the CaP (G) and stromal cells (H), in contrast to the lack of evident signal in the epithelium from normal saline injections (inlet of G) and in the BPH/PIN epithelium from the pCX-shh-IG injections (H). Hip-1 was detected in CaP (I), BPH/PIN (J), and in the normal saline-injected epithelium (inlet of I). Pten was detected in disperse cells within CaP (K) and in the epithelium from normal saline-injected prostates (inlet of K) as well as in the BPH/PIN epithelial cells (L).

FIG. 9 shows the HEDGEHOG, PATCH, GLI-1, GLI-2, GLI-3, and HIP protein expression in human prostate cancer tissues. Magnifications: A to H, 400×. HEDGEHOG expression detected by N-19 correlated with progression of prostate cancer, being almost absent in normal luminal epithelium (arrowheads in B) with more intense signals in the BPH (arrows in B) and PIN (asterisk in A, B) and most intense signals in the CaP (B and C). PATCH (D), GLI-1 (E), GLI-2 (F), GLI-3 (G), and HIP (H) were all highly expressed in the human CaP.

### DETAILED DESCRIPTION OF THE INVENTION

While the description sets forth various embodiment specific details, it will be appreciated that the description is illustrative only and should not to be construed in any way as limiting the invention. Furthermore, various applications of the invention, and modifications thereto, which may occur to those who are skilled in the art, are also encompassed by the general concepts described below.

Despite these previously published data, there is so far no animal model of prostate cancer caused initially by in vivo Hedgehog dysregulation from a normal status and in the prostate itself. Hence, a potential role of Hedgehog in the initiation of prostate cancer remains to be elucidated. In this invention, we addressed the effects of Hedgehog overexpression by introducing directly a Hedgehog-expressing vector into normal prostates. Preferably, the animal model of the invention is prepared simply by intra-prostatic injection, rather than conventional transgenic method. Thus, it can be easily established within a short time.

The present invention relates to an adult mammal which exhibits growth or replication of abnormal cells in a target tissue or organ by over-expressing Hedgehog protein in such target tissue or organ. The adult mammal is susceptible to cancer, preferably prostate cancer. The target tissue or organ is preferably a prostate, more preferably anterior or dorsolateral prostate.

In one embodiment, the adult mammal is produced by electroporation and/or intra-prostate injection with a Hedgehog-expressing vector.

In one embodiment, the Hedgehog protein is selected from the group consisting of Sonic Hedgehog (SHH), Desert Hedgehog (DHH), Indian Hedgehog (IHH), Echidna Hedgehog (EHH) and Tiggywinkle Hedgehog (TwHH), preferably Sonic Hedgehog (SHH).

Said adult mammal is preferably a mouse or a rat.

In one embodiment, said adult mammal exhibits a phenomenon associated with prostate cancer selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

In one embodiment, said adult mammal exhibits elevated expression level of a gene involved in Hedgehog signaling pathway selected from the group consisting of Ptc-1, Ptc-2, Gli-1, Gli-2, Gli-3, Smo and Hip.

The present invention also relates to a method of preparing an adult animal model of prostate cancer, comprising: (a) introducing a Hedgehog-expressing vector into a prostate of the animal; and (b) expressing the Hedgehog protein in the animal. The introduction of the Hedgehog-expressing vector is preferably conducted by electroporation and/or intra-prostate injection.

In one embodiment, the Hedgehog protein is selected from the group consisting of Sonic Hedgehog (SHH), Desert Hedgehog (DHH), Indian Hedgehog (IHH), Echidna Hedgehog (EHH) and Tiggywinkle Hedgehog (TwHH), preferably Sonic Hedgehog (SHH).

Said adult animal model is preferably a mouse or a rat.

In one embodiment, said adult animal model exhibits a phenomenon associated with prostate cancer selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

In one embodiment, said adult animal model exhibits elevated expression level of a gene involved in Hedgehog signaling pathway selected from the group consisting of Ptc-1, Ptc-2, Gli-1, Gli-2, Gli-3, Smo and Hip.

The invention further relates to a method of evaluating an agent for treating prostate cancer, comprising: (a) administering the agent to be evaluated to an adult animal model of prostate cancer which over-expresses Hedgehog protein in the prostate thereof; and (b) determining the effect of said agent upon a phenomenon associated with prostate cancer.

In one embodiment, said adult animal model exhibits a phenomenon associated with prostate cancer selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

In one embodiment, said adult animal model exhibits elevated expression level of a gene involved in Hedgehog signaling pathway selected from the group consisting of Ptc-1, Ptc-2, Gli-1, Gli-2, Gli-3, Smo and Hip.

The term “prostate cancer,” as used herein, refers to a malignant tumor of glandular origin in the prostate gland. According to the invention, the mouse of prostate cancer exhibits prostate intraepithelial neoplasia (PIN) and benign prostatic hyperplasia (BPH), as well as stromal hyperplasia under immunohistochemical detection.

The term “non-human animal,” as used herein, refers to an animal other than human. Preferably, the non-human animal is a mammal. More preferably, the non-human animal is a mouse.

The term “Hedgehog-expressing vector,” as used herein, refers to a vector that harbors a hedgehog family insert and can express Hedgehog protein in prostates in excessive amounts as observed by immunohistochemial detection. The Hedgehog protein is preferably selected from the group consisting of Sonic Hedgehog (SHH), Desert Hedgehog (DHH), Indian Hedgehog (IHH), Echidna Hedgehog (EHH) and Tiggywinkle Hedgehog (TwHH), more preferably Sonic Hedgehog (SHH).

The term “electroporating” or “electroporation” as used herein, refers to a technique by use of strong, brief pulses of electric current to create temporary holes in cell membranes, which allows the introduction of DNA into cells. In one preferred embodiment of the invention, the electric stimulation of electroporation is conducted for 10 seconds at 20 volts, 1 to 2 pulses per second.

According to the invention, PIN formation in the mouse model can be found at as early as 7 days after injection of Shh-expressing vector. Furthermore, prostate carcinoma can be found in the mouse model within 30 days after injection of Shh-expressing vector.

According to the invention, the Shh-expressing vector is injected into either anterior prostate (AP) or dorsolateral prostate (DLP) of a mouse. BPH and PIN can be found in either AP or DLP of the mouse of the invention.

As stated above, Hedgehog signaling pathway has been considered being relevant to prostate tumorigenesis. Moreover, a variety of Hedgehog signaling inhibitors have been under development to act as potential cures for tumors caused by Hedgehog dysregulation (M. Pasca di Magliano, M. Hebrok, Nat Rev Cancer 3, 903 (2003)). Thus, the gene expression involved in Hedge signaling pathway can used as the markers of detection on prostate cancer. According to the invention, the mouse model exhibits elevated expression level of a gene involved in Hedgehog signaling pathway selected from the group consisting of Ptc-1, Ptc-2, Gli-1, Gli-2 and Gli-3 by RT-PCR and immunohistochemical detection.

So far, transgenic approaches to establish Hedgehog over-expression animal model have failed, due to early death in uterus or in early post-natal periods. Known mouse models of prostate cancer are all started with cancer cells transplanted into mice, such as xenotransplanation of human prostatic carcinoma cells into nude mice. The mouse model of prostate cancer starts from normal status, which means Hedgehog protein can initiate prostatic carcinoma from normal cells, covering the entire process of prostate tumorigenesis can carcinogenesis from normal and benign stages to aggressive malignancy formation. According to the invention, a Hedgehog-induced prostate cancer mouse model can be easily established. BPH, PIN as well as prostatic carcinoma formation by Hedgehog protein expression is successfully induced, and the presence of Hedgehog protein is confirmed in correlation with early stages of prostate tumorigenesis. The Hedgehog-induced prostate tumorigenesis is further confirmed by commonly used immunohistochemical detection and serum markers. Furthermore, activation of Hedgehog signaling pathway during prostate tumorigenesis is confirmed by the alterations of its downstream signaling members and its interaction protein.

Thus, the mouse model of the invention with high efficiency of production and fast cancer progression from benign to malignant stages, and with major molecular characteristics, will be advantageous to screening therapeutic drug affiliated with prostate cancer as well as basic research on prostatic tumorigenesis can carcinogenesis.

### EXAMPLE

### Example 1

### Construction of a Mouse Expressing Hedgehog Protein

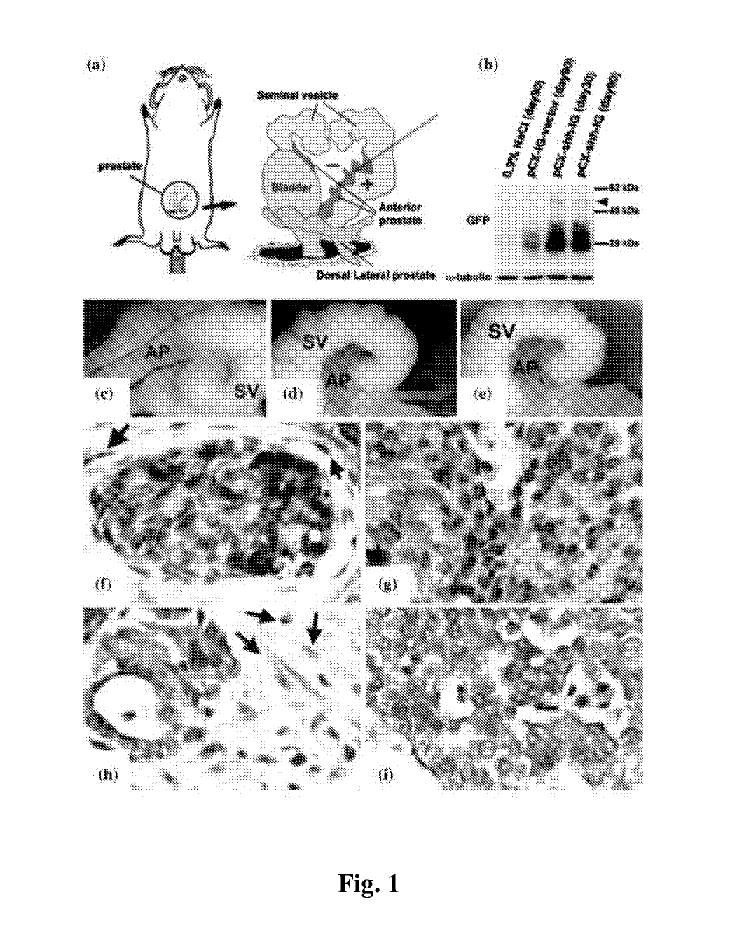

The Shh expression and vehicle vector, pCX-shh-IG and pCX-IG, were provided by Dr. Kerby C. Oberg, Loma Linda University. Male outbred FVB strain mice aged 8 to 10 weeks purchased from National Laboratory Animal Center, Academia Sinica, Taipei, were used for the injections. The mice were anesthetized with phenobarbital and exposed of their prostate glands by surgery (FIG. 1). For each injection, 10 μl of pCX-Shh-IG at 0.1 μg/μl in 0.9% NaCl was injected into the anterior lobe alone or into both the anterior and the dorsolateral lobes. Parallel injections with 10 μl of pCX-IG in 0.9% NaCl or 0.9% NaCl alone were performed as controls. After injections, the prostate lobes were subjected to electric stimulations (electroporation) for 10 seconds set at 20 volts, 1 to 2 pulses per second, using a Digitimer DS7 Stimulator (Digitimer, Hertfordshire, England). After electroporation, the mice were caged and maintained until use. With the above procedures, Shh expression was induced in the mouse prostates and the expression for the following 90 days after injection was traced.

### Example 2

### Immunohistochemical Detection

To confirm the efficiency of the prostate cancer formation in the mouse model, immunohistochemical detection was conducted as stated below. Tissue were dissected, fixed in 4% paraformaldehyde in PBS (Sigma), and processed to obtain 7 μm thick sections following standard histological preparations. For Gli-1, Gli-2, Gli-3, Hip detections, the sections were processed through citrate buffer (pH 2.0) for 4 min, followed by another citrate buffer (pH9.0) for 5 min and thorough washes before antibody binding. For CK14, p63, GFP, E-cadherin, N-19, 5E1, Ptc-1, Fgfr-2, and Fgf-2, the sections were treated with citrate buffer (pH 6) for 10 min before antibody binding. For Fgfr-1, Fgf-7, and Fgf-10, proteinase K (10 μg/ml) treatment was performed on ice for 5 min before processing through citrate buffer (pH 8.0) and antibody binding. The slides were then covered with antibody solutions at 4° C. overnight, then further processed with biotinylated secondary antibodies, followed by localization of immunoreactivity using the ABC immunoperoxidase method. All results were repeated in triplicate for confirmation. Antibodies for CK14 (sc-17104), Shh (N-19; sc-1194), Ptc-1 (G-19; sc-6149), Gli-1 (sc-6153), Gli-2 (sc-20290), Gli-3 (sc-6155), Hip (sc-9408), GFP (sc-9996), E-cadherin (sc-7870) were purchased from Santa Cruz Biotechnology Inc, Santa Cruz, Calif., USA. Antibodies for p63 (#MS-1801-P1), Pten (#RB-072-P1), were purchased from Lab Vision NeoMarkers, Fermont, Calif., USA. 5E1 anti-Shh antibodies were purchased from Developmental Studies Hybridoma Bank, Iowa City, Iowa, USA. Biotinylated secondary antibodies were obtained from Amersham International (Arlington Heights, Ill., USA). Peroxidase linked avidin/biotin complex reagents and the ABC immunoperoxidase kits were purchased from Vector Laboratories (Burlingame, Calif., USA).

### Example 3

### Confirmation of Hedgehog Overexpression in the pCX-shh-IG Injections

To solidify any data obtained as a result of Hedgehog overexpression, we examined Hedgehog expression status after the manipulation. We first examined the presence of GFP signals in wholemount preparations and in tissue sections. When both methods showed no convincing signal, Western analysis was used as a double check. In wholemounts (data not shown), GFP signals were detected in 15 prostates in a total of 25 pCX-shh-IG injections and in 8 of the 10 pCX-IG injections, but in none of the 0.9% NaCl blank controls. With immunohistochemistry, 23 of the 25 pCX-shh-IG injections and 8 of the 10 pCX-IG injections exhibited definite signals for GFP, with no positive signal detected in the 0.9% NaCl injections (Table 1). The two pCX-shh-IG injections without definite GFP signal in tissue sections were double checked using Western analyses (FIG. 1b) and RT-PCR (data not shown); both were found positive for GFP. We then checked by immunohistochemistry using anti-Shh and confirmed that all pCX-shh-IG injections positive for GFP were also evidently positive for Hedgehog protein expression, whereas no comparable signal was detected in the pCX-IG vehicle controls or the 0.9% NaCl blank controls. Therefore, the present data showed 100% (25/25) efficiency in introducing a Hedgehog overexpression status in the pCXshh-IG-injected prostates. Furthermore, the data demonstrated sustaining Hedgehog expression up to 90 days after the manipulation (Table 1 and FIG. 1b).

TABLE 1
Results of pCX-shh-IG, pCX-IG and 0.9% NaCl injections on detection

| | | Prostate | GFP as detected | Observation | Change in | Status of |
|---|---|---|---|---|---|---|
| | | injection site | by IHC | with HE-stain | stroma | mice |
| | Days after | | | | | |
| | pCX-shh-IG | | | | | |
| | injection | | | | | |
| 1. | Day7 | AP, DLP | + | PIN | Stroma ↑ | Death |
| 2. | Day7 | AP | + | PIN | Stroma ↑ | Death |
| 3. | Day20 | AP, DLP | + | PIN, CaP | Stroma ↑ | |
| 4. | Day20 | AP, DLP | + | PIN | ND | Death |
| 5. | Day30 | AP, DLP | +/− | PIN | ND | |
| 6. | Day30 | AP, DLP | + | PIN, CaP | Stroma ↑ | |
| 7. | Day30 | AP, DLP | + | PIN | Stroma ↑ | |
| 8. | Day30 | AP, DLP | + | PIN, CaP | Stroma ↑ | |
| 9. | Day30 | AP | +/− | PIN | Stroma ↑ | |
| 10. | Day30 | AP | + | PIN | Stroma ↑ | Death |
| 11. | Day30 | AP | + | PIN | ND | |
| 12. | Day30 | AP | + | PIN, CaP | Stroma ↑ | |
| 13. | Day30 | AP | + | PIN | Stroma ↑ | |
| 14. | Day30 | AP | + | PIN, CaP | ND | |
| 15. | Day30 | AP, DLP | + | PIN | Stroma ↑ | |
| 16. | Day30 | AP, DLP | + | PIN, CaP | Stroma ↑ | |
| 17. | Day30 | AP, DLP | + | PIN | Stroma ↑ | |
| 18. | Day30 | AP | + | PIN | Stroma ↑ | |
| 19. | Day30 | AP | + | PIN | ND | |
| 20. | Day90 | AP, DLP | + | PIN, CaP | Stroma ↑ | |
| 21. | Day90 | AP, DLP | + | PIN | Stroma ↑ | |
| 22. | Day90 | AP, DLP | + | PIN | Stroma ↑ | |
| 23. | Day90 | AP | + | PIN, CaP | Stroma ↑ | |
| 24. | Day90 | AP | + | PIN, CaP | Stroma ↑ | |
| 25. | Day90 | AP | + | PIN | Stroma ↑ | |
| | Days after | | | | | |
| | pCX-IG | | | | | |
| | injection | | | | | |
| 1. | Day20 | AP, DLP | + | Normal | ND | |
| 2. | Day20 | AP, DLP | + | Normal | ND | |
| 3. | Day30 | AP, DLP | + | Normal | ND | |
| 4. | Day30 | AP | + | Normal | ND | |
| 5. | Day30 | AP | + | Normal | ND | Death |
| 6. | Day30 | AP | + | Normal | ND | |
| 7. | Day30 | AP | +/− | Normal | ND | |
| 8. | Day90 | AP, DLP | +/− | Normal | ND | |
| 9. | Day90 | AP, DLP | + | Normal | ND | |
| 10. | Day90 | AP | + | Normal | ND | |
| | Days after | | | | | |
| | 0.9% NaCl | | | | | |
| | injection | | | | | |
| 1. | Day20 | AP, DLP | — | Normal | ND | |
| 2. | Day20 | AP, DLP | — | Normal | ND | |
| 3. | Day30 | AP, DLP | — | Normal | ND | |
| 4. | Day30 | AP, DLP | — | Normal | ND | |
| 5. | Day30 | AP | — | Normal | ND | |
| 6. | Day30 | AP | — | Normal | ND | |
| 7. | Day30 | AP | — | Normal | ND | |
| 8. | Day90 | AP, DLP | — | Normal | ND | |
| 9. | Day90 | AP, DLP | — | Normal | ND | |
| 10. | Day90 | AP | — | Normal | ND | |

AP, anterior prostate;
DLP, dorsolateral prostate;
CaP, prostate cancer;
GFP, green fluorescent protein;
HE-stain, Hematoxylin-Eosin stain;
IHC, immunohistochemistry;
PIN, prostate intraepithelial neoplasia;
ND, not detected.

### Example 4

### Gross Morphological Effects with Hedgehog Overexpression in the Prostates

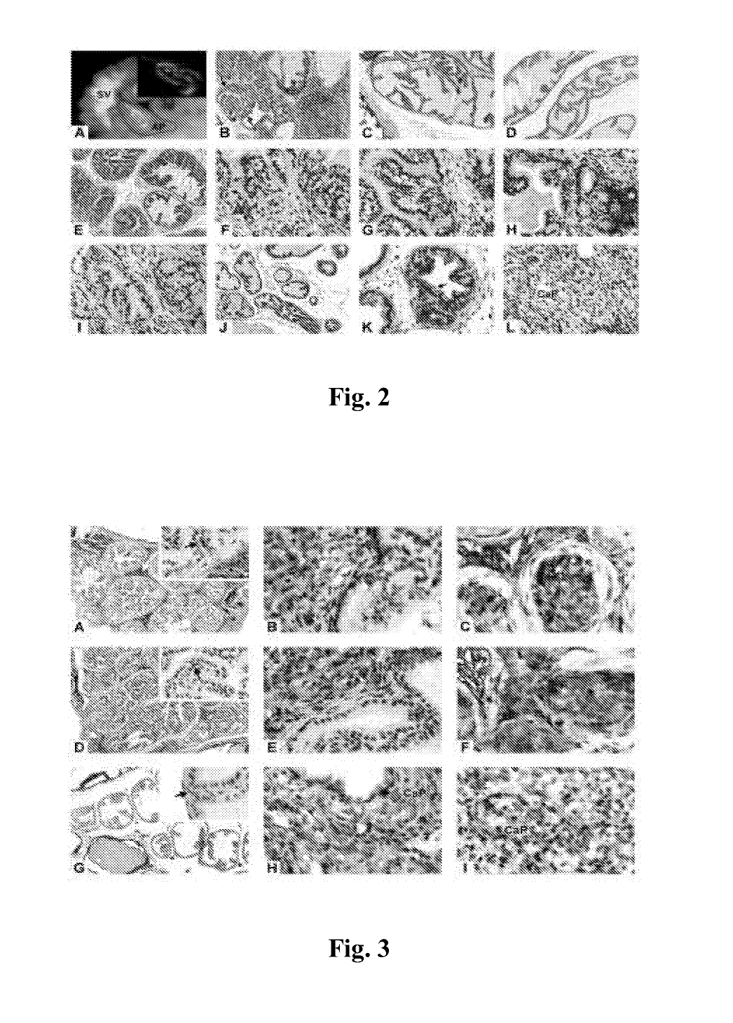

Gross morphological effects were examined at days 7, 20, 30, and 90 after the manipulation and the most prominent changes were found at day 30 so far. By day 30 after the injection, the anterior prostates (AP) of the pCX-shh-IG injections exhibited overgrowth (FIG. 1c), in contrast to those seen in the pCX-IG vehicle controls (FIG. 1d) or in the 0.9% NaCl blank controls (FIG. 1e). We assumed that the overgrowth in the pCX-shh-IG-injected prostates was due to the effects of Hedgehog overexpression, since no comparable result was found in the two control groups. In addition, seminal vesicles (SV) in the pCX-shh-IG-injected prostates showed less evident lobular formation as compared to those in the pCX-IG and the 0.9% NaCl injections (FIG. 1c vs. FIGS. 1d and e). The size of seminal vesicles appeared to enlarge with pCX-shh-IG injections. Furthermore, hypervascularization in the prostates was observed in wholemount preparations FIG. 1c) as well as in tissue sections (FIG. 2d).

### Example 5

### Mouse Hedgehog Overexpression Patterns were Comparable to Human Conditions

Conceivably, any animal model should be reflective of human conditions so that data from animal analyses could be applicable to human diseases. To solidify that the mouse prostates with Hedgehog overexpression could be used as study models for the human conditions, we compared the pCXshh-IG-injected mouse prostates with the human CaP specimen in their Hedgehog expression patterns by immunohistochemistry. Activation of Hedgehog was seen in 38 out of the 40 human specimen (FIGS. 1h and i), being in both the epithelial and the stromal cells, and the expression patterns were comparable to those observed in the mouse (FIGS. 1f and g). The comparable Hedgehog overexpression patterns in the mouse indicated phenocopying of the human status.

### Example 6

### Detection of the Efficiency of the Mouse Expressing Hedgehog Protein

The efficiency of the mouse prepared in Example 1 was examined by the immunofluorescence microscopy and the immunohistochemical detection against GFP at 7, 20, 30, and 90 days after injections. The results were compared and shown in Table 1 and FIG. 2.

GFP expression was detected in at least 23 out of 25 prostates injected with pCX-shh-IG (FIG. 2A and Table 1), in parallel with 8/10 of the pCX-GFP injections and in contrast to 0/10 of the normal saline injections. The efficiency was considered very satisfactory and thus the efficacy of Shh expression was further examined. It was found that 100% (25/25) of the prostates injected with pCX-shh-IG exhibited PIN (FIGS. 2B and E to K), irrespective of injection into either anterior (AP) or dorsolateral prostate (DLP). This was in contrast to the single PIN-like case of the 10 pCX-IG injections (FIG. 2C), and none of the 10 normal saline injections exhibited PIN (FIG. 2D). The pCX-shh-IG group also exhibited BPH along with PIN, and even three cases of CaP (prostate carcinoma) at day 30 after injection (FIG. 2L). The extensive stromal growth was also found in most of the pCX-shh-IG injections (Table 1; FIGS. 2B and E to L), but not in the vehicle or normal saline injections (FIGS. 2C and D). Noticeably, the pCX-shh-IG injections caused PIN formation at as early as day 7 after the procedures, which was faster than any other mouse models that had been reported that transformed normal prostate epithelium into neoplasia under in vivo conditions. Moreover, no comparable PIN formation was found in the pCX-IG or the normal saline injections, which indicated that the PIN formation in the pCX-shh-IG group was less likely due to acute inflammatory response to the injection or the electroporation procedures.

### Example 7

### Confirmation of the Presence and Distribution of Shh Expression

GFP was presumed to be the marker of functional Hedgehog protein. Therefore, the presence of GFP was further examined in the three injection groups by Western analysis to confirm the aforementioned fast efficiency caused by the Shh expression.

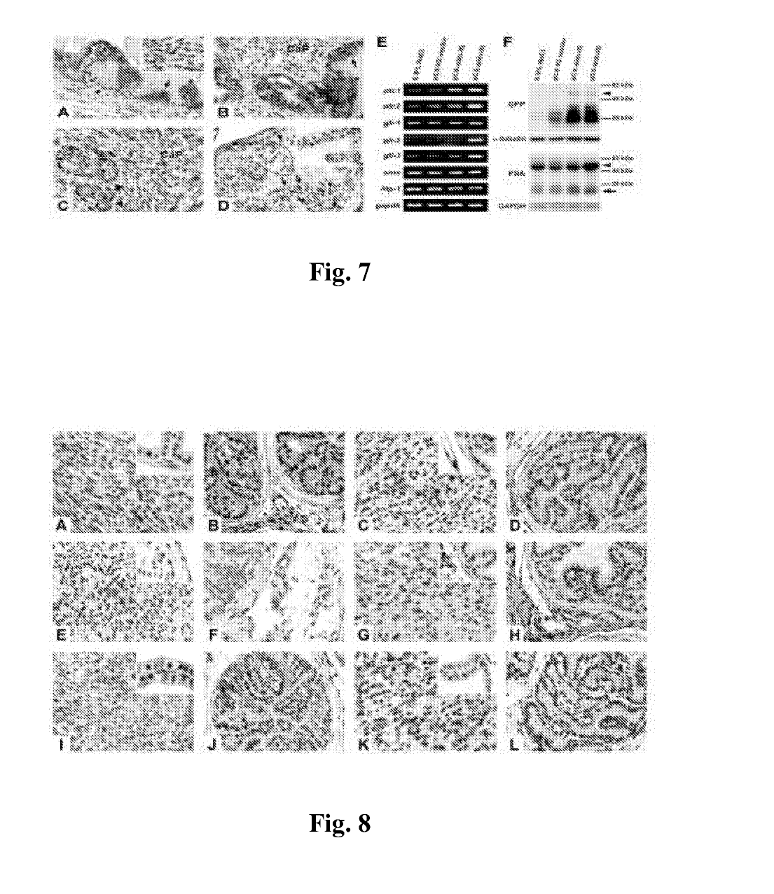

The results of the Western analyses with anti-GFP antibody showed the presence of Hedgehog protein tagged with GFP in the pCX-shh-IG group, but not in the pCX-IG and 0.9% NaCl saline injections (upper panel of FIG. 7F; indicated by arrowhead).

Thereafter, the GFP distribution was further examined and correlated with the sites in which Hedgehog protein could be detected by 5E1 anti-Shh antibody. At the day 30 after injection, GFP was localized extensively in the stromal cells of the prostate injected with pCX-shh-IG (FIG. 3A). GFP was also found within the basal cells (inlet of FIG. 3A), comparable to that detected by 5E1 (inlet of FIG. 3D) in the pCX-shh-IG injections, and to that seen in the normal saline injections detected by 5E1 (inlet of FIG. 3G). In the luminal epithelium, evident signals of GFP were more commonly detected in the high grade PIN (FIG. 3C), as compared to those in the low grade PIN (FIG. 3B). The distribution of GFP matched well with the localization of Hedgehog protein, as shown by immunohistochemical detection using 5E1 anti-Shh antibody (FIGS. 3D to F). Abundant Hedgehog protein was detected with concurrent stromal hyperplasia or presence of CaP (FIGS. 3H, I), which was not seen in the pCX-IG vehicle controls (not shown), nor in the normal saline injections (FIG. 3G).

### Example 8

### PIN and CaP Formation with Hedgehog Overexpression

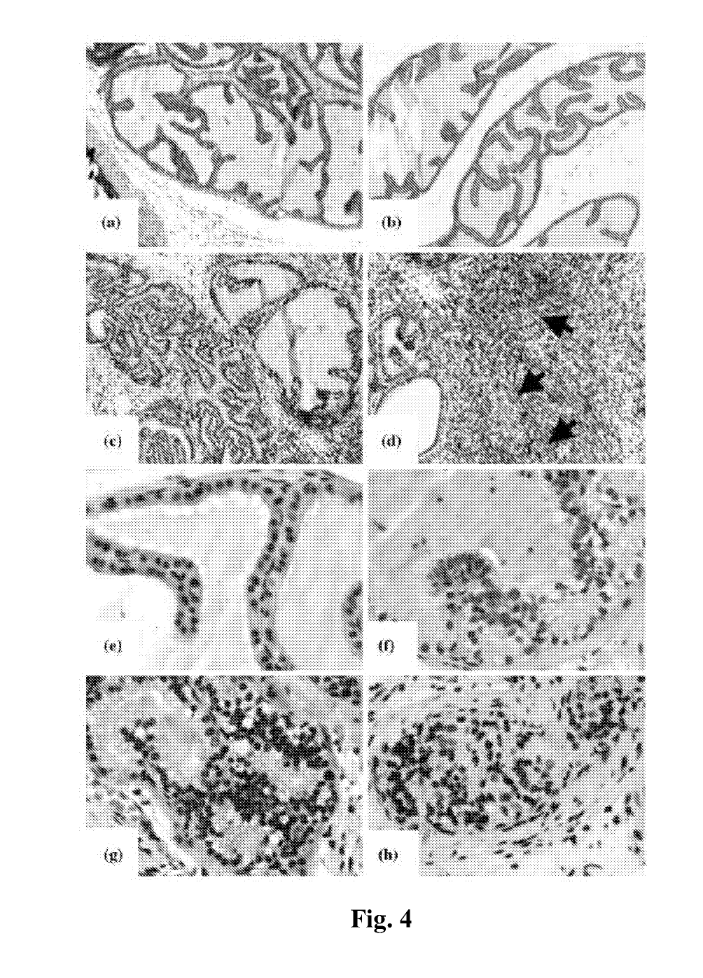

The overgrowth with Hedgehog overexpression was further analyzed microscopically to elucidate whether prostate tumorigenesis had occurred. Prostatic intraepithelial neoplasia (PIN) was found in all pCX-shh-IG-injected prostates (25/25) at 7, 20, 30 and 90 days after pCX-shh-IG injections (Table 1; FIGS. 1f and g, 4c, f-h). Unlike the conditions in the 0.9% NaCl-injected (FIG. 4a) and the pCX-IG-injected controls (FIG. 4b), the pCX-shh-IG-injected prostate tissue sections showed infolded glands with stromal hyperplasia (FIG. 4c) and often also with hypervascularization (FIG. 4d). Derangements of epithelial cells were found in the pCX-shh-IG-injected prostates (FIGS. 1f and g, 4f-h), but not in the controls (FIG. 4e). The derangements and infoldings, we believe, were not due to artifacts of oblique slicing, since the atypical cells were often seen in multifocal sites and were heterogeneous to surrounding typical epithelial glands within a same tissue (FIG. 4c). For the same reason, we believe that the observed abnormalities were not due to non-specific acute inflammatory responses to the injection and electroporation procedures. These heterogeneous and multifocal characteristics also indicated phenocopying of the human tumorigenesis.

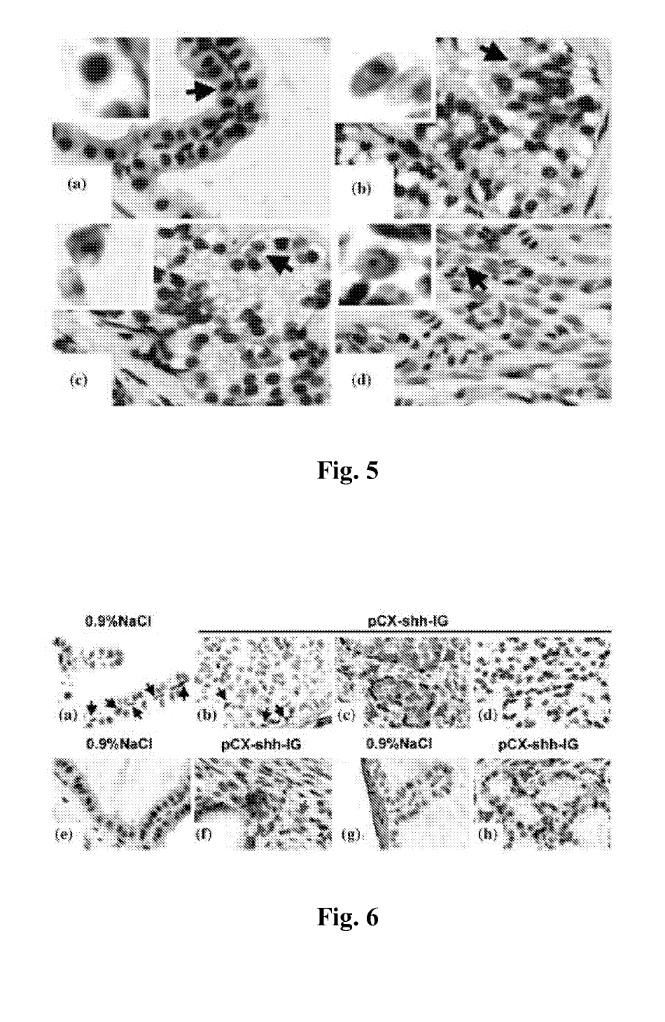

In humans, high grade PIN is the believed precursor of CaP. Both high grade PIN and CaP, compared to normal prostate glands, have enlarged nuclei with prominent nucleoli. PIN is characterized by large infolded glands surrounded by a discontinuous layer of basal cells, whereas CaP glands are smaller and lack basal cells. To confirm PIN and CaP formation in the present mouse Hedgehog overexpression model, these microscopic phenotypes were examined. With higher magnification, the epithelial cells with Hedgehog overexpression showed enlarged nuclei with prominent nucleoli, typical of high grade PIN (FIG. 5b) and CaP (FIGS. 5c and d) formations; these atypical cells were not found in the 0.9% NaCl blank controls or the pCX-IG vehicle controls (FIG. 5a). Further indications of PIN formation, as it was distinguished from CaP, were the loss of basal cell continuity confirmed by p63 immunodetection (FIG. 6a vs. b). It appeared that the basal cells began to lose their immunoreactivity to p63 antibody as the epithelial cells became invasive (FIG. 6c). When CaP was formed, no evident p63 immunoreactivity was detected (FIG. 6d). The invasive CaP formation was supported by invasion of CK-8/CK-18 positive epithelial cells into the stroma (FIG. 6f), in contrast to the normal glandular positioning in the controls (FIG. 6e). We also found thinner and disrupted distribution of fibro-muscular cell marker α-SMA in the pCX-shh-IG-injected prostates (FIG. 6h), indicating muscle layer disruption and epithelial invasion. This disruption of fibromuscular sheath was not found in the controls injected with 0.9% NaCl (FIG. 6g).

Based on these criteria, CaP formation was observed in 9 cases (9/25) after pCX-shh-IG injections (Table 1). All of the observed PIN and CaP formations were not found in the pCX-IG vehicle (0/10), nor in the 0.9% NaCl controls (0/10). These results indicated that the characteristics of PIN and CaP formation were specifically due to Hedgehog overexpression, instead of the procedures of injections or electroporations.

### Example 9

### Confirmation of Prostatic Tumorigenesis

In order to confirm the prostatic tumorigenesis, immunohistochemical detection using E-cadherin, CK14, and p63 as markers, as well as Western analysis of serum prostatic specific antigen (PSA) as described above were conducted. The results were elucidated as below.

E-cadherin was intensely expressed in the PIN, with much less expression along the lateral membrane of normal and BPH luminal cells (FIG. 7A). Within the area of CaP, E-cadherin signals were diminished (inlet of FIG. 7A). Similarly, the basal cell marker CK14 was intensely expressed in a manner of displacement and derangement in the BPH and PIN and was diminished within the area of CaP (FIG. 7B). Whereas, another basal cell marker p63 was highly expressed within PIN and CaP (FIG. 7C), as compared to that within BPH (FIG. 7D). Since both CK14 and p63 were basal cell markers and were only sparsely detected in the normal saline-injected prostates (inlet of FIG. 7D), Hedgehog protein expression might induce basal cell hyperplasia and transformation. Western analysis further confirmed the histological finding of prostate tumorigenesis by showing increased PSA secretion into serum (lower panel of FIG. 7F).

### Example 10

### Detection of Hedgehog Signaling Pathway

RT-PCR and immunohistochemical detection were conducted to examine the expression of Ptc-1, Ptc-2, Gli-1, Gli-2, Gli-3, Smo, and Hip, which are the members of Hedgehog signaling pathway. If Hedgehog protein expression was responsible for the prostate tumorigenesis, the members of its signaling pathway had to be expressed to constitute a functional activation. Thus, the activation of these genes can solidify the above observed effects of Hedgehog expression in the mouse model of the invention.

Total RNA was isolated from prostates by using the TRIzol method (Life Technologies) and prepared at 2 μg/μl. RT was run for 2 hours in a 100 μl reaction mixture containing 60 μl of Depc-treated H₂O, 20 μl of 5× reaction buffer, 6 μl of total RNA, 8 μl of dT at 0.5 mg/ml, 5 μl of 10 mM dNTP, and 1 μl of MMLV reverse trancriptase (200 units). PCR was run in a 50 μl reaction mixture containing 36 μl of Depc-treated H₂O, 5 μl of 10× reaction buffer, 5 μl of cDNA, 1 μl of 200 mM dNTPs, 1 μl of each sense and anti-sense 10 μM primers, and 0.25 μl of Tag Polymerase.

The sequences of the primers used in RT-PCR were based on the following publications: Ptc-1, Ptc-2, Gli-1, Gli-3, Smo (The FASEB Journal express online article 10.1096/fj.03-0293fje, 2003); Gli-2 (Develop Biol, 249:349-366, 2002); Hip-1 (Development 130:4871-4879, 2003).

All the RT-PCR reagents and primers were purchased from Life Technologies, Carlsbad, Calif., USA. The running parameters were: a 95° C. start for 5 min, followed by 35 cycles of 95° C. for 1 min, annealing temperature for 50 sec (Ptc-1 at 60° C., Ptc-2 at 57° C., Gli-1 at 60° C., Gli-2 at 57° C., Gli-3 at 57° C., Smo at 58° C., Hip at 57° C.), 72° C. for 1 min, and ended by 72° C. for 7 min.

The RT-PCR analyses using total prostate RNA preparations showed elevated Ptc-1, Ptc-2, Gli-1, Gli-2, and Gli-3 expression in the pCX-shh-IG injections, whereas Smo and Hip expression appeared not affected (FIG. 7E). The immunohistochemical detection showed Ptc-1 expression in the CaP (FIG. 8A) at a relatively higher level than that in the normal saline-injected luminal epithelium (inlet of FIG. 8A). Ptc-1 expression was also detected in the BPH/PIN epithelial cells and the stromal cells (FIG. 8B), where the signals appeared to be the same or even more intense than those in the CaP. Gli-1 was highly expressed in the CaP (FIG. 8C), the BPH/PIN epithelial cells, and the stromal cells (FIG. 8D), in contrast to the absence of signal in the epithelium from normal saline injections (inlet of FIG. 8C). Gli-2 expression was found in the CaP (FIG. 8E), but not in the epithelium from normal saline injections (inlet of FIG. 8E). Different from Gli-1 expression, however, Gli-2 seemed not expressed in the BPH/PIN epithelial cells of the anterior lobe (FIG. 8F), but was intensely expressed in the dorsolateral lobe (data not shown). Similar to Gli-1 and Gli-2, Gli-3 was expressed in the CaP (FIG. 8G), in contrast to the absence of signal in the epithelium from normal saline injections (inlet of FIG. 8G). Like Gli-1, Gli-3 was also highly expressed in the stromal cells, but no evident Gli-3 signal was detected in the BPH/PIN epithelium from the pCX-shh-IG injections (FIG. 8H). Hip was detected in the CaP (FIG. 81) at a level much less than that in the normal saline-injected luminal epithelium (inlet of FIG. 81). Within the BPH/PIN, Hip was stained (FIG. 8J). The Hedgehog signaling pathway in the three injection groups were summarized in the following Table 2.

TABLE 2
Results of RT-PCR detection on Hedgehog signaling pathway

| | | | | |
|---|---|---|---|---|
| Ptc-1 | Gli-1 | Gli-2 | Gli-3 | Hip |

Normal prostate (0.9% NaCl and pCX-IG vector)

| | | | | | |
|---|---|---|---|---|---|
| Luminal epithelium | + | ND | ND | ND | +++ |
| Stroma | + | + | ND | + | + |

Tumor prostate (pCX-shh-IG vector)

| | | | | | |
|---|---|---|---|---|---|
| Luminal epithelium | | | | | |
| BPH/PIN | +++ | ++ | (++++)* | ND | + |
| CaP | +++ | ++++ | ++ | ++++ | + |
| Reactive Stroma | +++ | ++ | ++ | +++ | + |

*only seen in dorsolateral lobes
ND: not detected

### Example 11

### Detection of Tumor Suppressor Gene Pten

Pten is a tumor suppressor gene that has been implicated in the formation of prostate carcinoma and several other carcinomas with loss-of-function (A. Di Cristofano, P. P. Pandolfi, Cell 100, 387 (2000)). Therefore, Pten was further examined in the invention. As a result, Pten was detected in disperse cells within CaP (FIG. 8K) and appeared to be expressed in the epithelium from normal saline-injected prostates (inlet of FIG. 8K). Pten expression was also found in the BPH/PIN epithelial cells (FIG. 8L).

### Example 12

### Comparison of the Prostate Cancer of the Mouse Model of the Invention and that of Human Cancer Tissue

In order to access the similarity of prostate cancer of the mouse model as prepared above to human prostate cancer, the status of Hedgehog signaling in human prostate cancer tissues was examined, and the result was compared with that shown in the mouse model of the invention.

The human prostate tissues were obtained from patients undergoing radical prostectomy or needle biopsy at Department of Pathology, Chung Shan Medical University The presence of CaP was confirmed by histological examination. All procedures of tissue procurement and experiments were reviewed and approved by the IRB of Chung Shan Medical University.

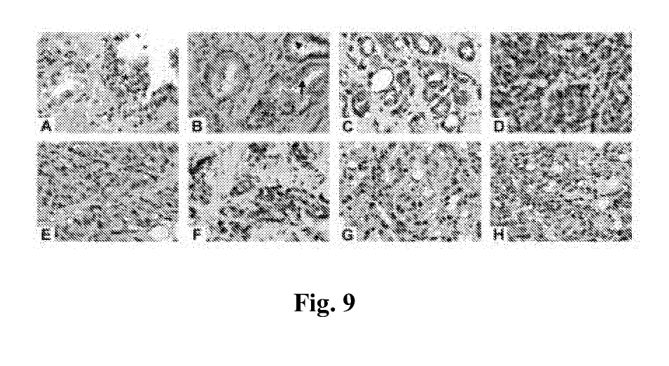

Immunohistochemical detection using N-19 anti-HEDGEHOG antibody showed distribution in the human prostate tissues, from the benign to the malignant status (FIGS. 9A to C). Similar to GFP and 5E1 (FIGS. 3C and F) as well as to CK-14 and p63 (FIGS. 7B and C) in the pCX-shh-IG injection samples, HEDGEHOG protein in human BPH-PIN samples was expressed in a manner of displacement and derangement. The expression of Ptc-1, Gli-1, Gli-2, Gli-3 (FIGS. 9D to G), and Hip (FIG. 9H) in the human CaP portrayed a general picture of activation. These data demonstrated that Hedgehog signaling members were activated during the progressing of the benign cells towards the aggressive malignant tumor cells, and such level of activation was absent in the normal prostates. Furthermore, the Hedgehog-induced mouse model matched well with the human conditions.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. An adult mammal which exhibits growth or replication of abnormal cells in a target tissue or organ by over-expressing Hedgehog protein in such target tissue or organ.

2. The adult mammal of claim 1, which is susceptible to cancer.

3. The adult mammal of claim 1, which is susceptible to prostate cancer.

4. The adult mammal of claim 1, wherein the target tissue or organ is a prostate.

5. The adult mammal of claim 4, which is produced by electroporation and/or intra-prostate injection with a Hedgehog-expressing vector.

6. The adult mammal of claim 1, wherein the Hedgehog protein is selected from the group consisting of Sonic Hedgehog (SHH), Desert Hedgehog (DHH), Indian Hedgehog (IHH), Echidna Hedgehog (EHH) and Tiggywinkle Hedgehog (TwHH).

7. The adult mammal of claim 6, wherein the Hedgehog protein is Sonic Hedgehog (SHH).

8. The adult mammal of claim 1, which exhibits a phenomenon associated with prostate cancer.

9. The adult mammal of claim 8, wherein the phenomenon associated with prostate cancer is selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

10. The adult mammal of claim 1, which exhibits elevated expression level of a gene involved in Hedgehog signaling pathway selected from the group consisting of Ptc-1, Ptc-2, Gli-1, Gli-2, Gli-3, Smo and Hip.

11. A method of preparing an adult animal model of prostate cancer, comprising
(a) introducing a Hedgehog-expressing vector into a prostate of the animal; and
(b) expressing the Hedgehog protein in the animal.

12. The method of claim 11, wherein introducing the Hedgehog-expressing vector is conducted by electroporation and/or intra-prostate injection.

13. The method of claim 11, wherein the Hedgehog protein is selected from the group consisting of Sonic Hedgehog (SHH), Desert Hedgehog (DHH), Indian Hedgehog (IHH), Echidna Hedgehog (EHH) and Tiggywinkle Hedgehog (TwHH).

14. The method of claim 13, wherein the Hedgehog protein is Sonic Hedgehog (SHH).

15. The method of claim 11, wherein the adult animal exhibits a phenomenon associated with prostate cancer.

16. The method of claim 15, wherein the phenomenon associated with prostate cancer is selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

17. The method of claim 11, wherein the adult animal exhibits elevated expression level of a gene involved in Hedgehog signaling pathway selected from the group consisting of Ptc-1, Ptc-2, Gli-1, Gli-2, Gli-3, Smo and Hip.

18. A method of evaluating an agent for treating prostate cancer, comprising:
(a) administering the agent to be evaluated to an adult animal model of prostate cancer which over-expresses Hedgehog protein in the prostate thereof; and
(b) determining the effect of said agent upon a phenomenon associated with prostate cancer.

19. The method of claim 18, wherein the phenomenon associated with prostate cancer is selected from the group consisting of benign prostatic hyperplasia (BPH), prostate intraepithelial neoplasia (PIN), prostatic cancer (CaP) phenotypes, prostatic stromal hyperplasia and enhanced angiogenesis of prostate.

20. The method of claim 18, wherein said determining the effect of said agent is conducted by detection on the expression level of a gene involved in Hedgehog signaling pathway selected from the group consisting of Ptc-1, Ptc-2, Gli-1, Gli-2, Gli-3, Smo and Hip.
